(19) United States
(12) Patent Application Publication
(10) Pub. No.: US 2007/0213759 A1
(43) Pub. Date: Sep. 13, 2007

(54) Puncture resistant balloon catheter

(76) Inventors: Thomas A. Osborne, Bloomington, IN (US); M. Kem Hawkins, Bloomington, IN (US)

Correspondence Address: BRINKS HOFER GILSON & LIONE, P.O. BOX 10395, CHICAGO, IL, 60610, US

(21) Appl. No.: 11/714,579

(22) Filed: Mar. 6, 2007

Related U.S. Application Data
(60) Provisional application No. 60/780,147, filed on Mar. 8, 2006.

(51) Int. Cl. A61M 29/00

(52) U.S. Cl. 606/192

(57) ABSTRACT

A puncture resistant balloon catheter device and a method of using the device is described. The device is a balloon catheter having a puncture resistant cover disposed over the balloon. The cover is capable of moving between a deflated state and an expanded state. The cover inhibits piercing of the balloon surface that may occur during delivery and deployment of a stent in a body lumen.

### RELATED APPLICATIONS

This application claims the benefit of priority from U.S. Provisional Application Ser. No. 60/780,147 filed Mar. 8, 2006, which is incorporated herein by reference.

### TECHNICAL FIELD

The invention generally relates to a balloon catheter having a puncture resistant covering.

### BACKGROUND

When a stent graft is implanted within a main body lumen having an aneurysm, the graft preferably does not occlude any side branch vessels. For example, if a renal artery or pulmonary artery is occluded by a stent graft, the blood supplied by these arteries to the vital organs would be stopped, thereby causing damage to the organ tissues. Accordingly, it is preferable that the stent graft include holes or fenestrations which are aligned with the side branch openings. Such alignment of the fenestration with the side branch enables blood to continue to flow into these branches.

The fenestration generally forms a tight seal with the side branched opening. A lack of a tight seal may cause blood to leak out of the stent graft and into the gap between the stent graft and main body lumen. Such leakage can cause the aneurysm in the main body lumen to continue to be pressurized. Accordingly, a small balloon expandable stent may be implanted within the side branch vessel to create a tight seal at the site of the fenestration and vessel.

Conventional balloon catheters may be used to maneuver through the fenestration of the stent graft and deploy a stent. However, conventional balloon catheters are prone to puncture during the delivery and deployment of the stent. For example, current fenestrations typically employ a rim of wire, which contacts the surface of the balloon and potentially results in damage and rupture of the balloon. Additionally, expansion of the balloon expandable stent typically involves the proximal end of the stent disposed within the stent graft. In order to connect the stent to the graft, the stent may be balloon expanded such that the struts at the proximal end of the stent will flare. However, this flaring may cause the struts to penetrate the balloon and puncture it.

In addition, many arteries contain calcified lesions that may be sharp. Expansion of such arterial walls require large dilation pressures that conventional balloon catheters may not possess. Furthermore, even if expansion of such calcified arterial walls is possible, the sharp calcified lesions may rupture the balloon, thereby requiring another balloon catheter to be inserted and the procedure repeated.

### SUMMARY

Accordingly, a punctured resistant balloon catheter is provided. Although the inventions described below may be useful for increasing the control, accuracy and ease of placement during deployment of the prosthesis, the claimed inventions may also solve other problems.

In a first aspect, a puncture resistant balloon catheter is provided comprising a catheter comprising a distal end, a shaft extending along a longitudinal axis of the catheter, and an inflation lumen extending therethrough. An inflatable balloon is disposed over the shaft of the catheter. A puncture resistant cover is disposed over the balloon. The cover extends circumferentially around the longitudinal axis of the catheter. The cover inhibits piercing of the balloon and is adapted to be movable between a deflated state and an inflated state.

In a second aspect, a method of breaking up calcified lesions within a body lumen is provided. A puncture resistant balloon catheter comprising a catheter, an inflatable balloon, and a puncture resistant cover disposed over the balloon is provided. A wire guide is fed through the patient's skin. The wire guide is then fed through a wire guide lumen of the catheter. The balloon catheter is advanced over the wire guide towards the body lumen having the calcified lesions. Upon reaching the calcified region, the balloon is inflated. Inflation of the balloon transforms the cover from the deflated configuration to an inflated configuration. The cover in the inflated configuration breaks up the calcified lesions, and the cover inhibits piercing of the balloon by the calcified lesions.

In a third aspect, a method of deploying within a branched body lumen a side branch balloon expandable stent through a fenestration of a graft is provided. A puncture resistant balloon catheter is provided comprising a catheter. The catheter comprises a distal end, a wire guide lumen and an inflation lumen extending therethrough. An inflatable balloon is disposed over the catheter. The balloon extends from the distal end of the catheter. A puncture resistant cover is disposed over the balloon. The cover extends circumferentially around the longitudinal axis of the catheter. The cover is adapted to be movable between a deflated state and an inflated state and the cover inhibits puncture of the balloon. A side branch balloon expandable stent is also provided. The stent is disposed over the puncture resistant cover. The puncture resistant balloon catheter is advanced over a wire guide. The balloon is in a deflated state. The puncture resistant balloon catheter is advanced into the graft, and the fenestration of the graft is aligned with the side branch vessel. The puncture resistant balloon catheter is then fed through the fenestration of the graft and into the branched body lumen. Fluid is passed through the inflation lumen to inflate the balloon. The inflation of the balloon causes the cover to transform from the deflated state to the expanded state. The cover exerts an outward force to expand the stent against one or more walls of the branched body lumen. In its expanded state, the stent has a distal end extending within the branched body lumen and a proximal end extending through the fenestration of the graft.

Additional details and advantages of the invention are described below and shown in the drawings.

### BRIEF DESCRIPTION OF THE DRAWINGS

Embodiments will now be described by way of example with reference to the accompanying drawings, in which:

FIG. 1 is a perspective view of a puncture resistant balloon catheter;

FIG. 2 is a blown-up perspective view of the puncture resistant balloon catheter of FIG. 1;

FIG. 3 is a cross-sectional view of the puncture resistant balloon catheter in an inflated configuration;

FIG. 4 is a cross-sectional of the puncture resistant balloon catheter in a deflated configuration;

FIG. 5 is a perspective view of a main lumen with an aneurysm and a healthy branch lumen;

FIG. 6 is a perspective view of a stent graft implanted in the aneurysm of the main lumen;

FIG. 7 is a perspective view of a balloon expandable stent implanted within the side branched body lumen;

FIG. 8 is a side view of the stent graft;

FIG. 9 is a blown up view of FIG. 8 showing the fenestration; and

FIG. 10 is a cross-sectional view taken along the longitudinal axis of the puncture resistant balloon catheter in an expanded state.

### DETAILED DESCRIPTION OF THE PREFERRED EMBODIMENTS

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

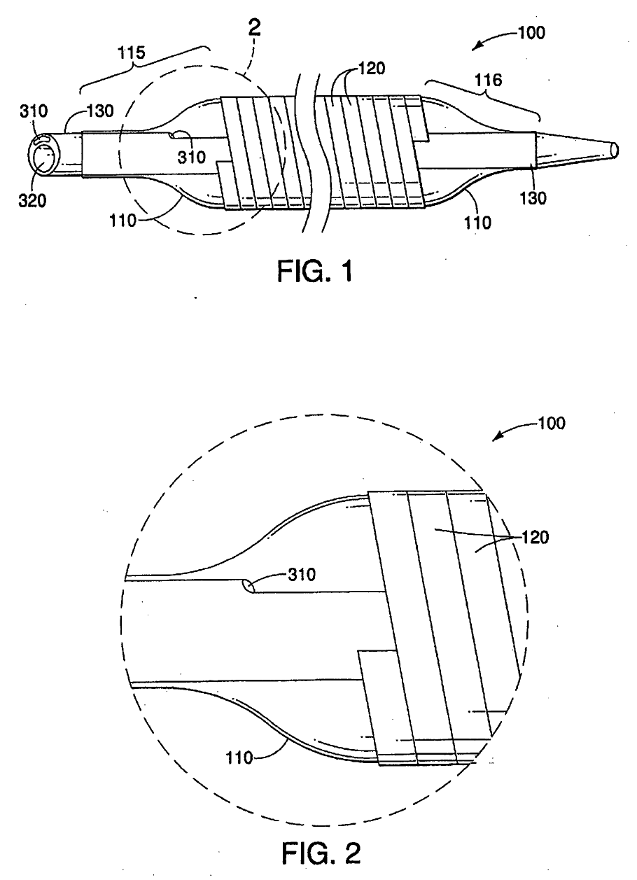

An exemplary puncture resistant balloon catheter 100 is shown in FIG. 1. FIG. 1 shows the balloon catheter 100 with the balloon 110 in its expanded state. The balloon 110 is disposed over the catheter 130 and extends along the longitudinal axis of the catheter 130. The visible portion of the balloon 110 is shown with tapered ends 115 and 116. The tapered ends 115 and 116 extend outward from within the armored ribbon coil 120 toward the surface of the catheter 130. A blown-up view of tapered end 115 is shown in FIG. 2. The balloon 110 may be formed from any suitable polymeric material known to those of ordinary skill in the art, including polyethylene terephthalate (PET) and nylon.

The majority of the balloon 110 may be secured within an armored coil 120, as shown in FIGS. 1 and 2. The armored coil 120 may be a puncture resistant covering that may be used to protect the balloon surface from inadvertent puncture during delivery and deployment of a balloon expandable stent within a fenestrated stent graft. The procedure will be described in more detail below.

The armored coil 120 is shown in FIGS. 1 and 2 as a ribbon coil that overlies the balloon 110. The ribbon material may be any suitable puncture resistant material, including stainless steel, nitinol, and palladium. The thickness and width of the armored coil 120 may be dependent upon a variety of factors, including the type of balloon and catheter utilized. In this example, the ribbon material preferably has a thickness ranging from about 0.0001 inches to about 0.0020 inches. The ribbon material preferably has a width ranging from about 0.010 inches to about 0.040 inches. Generally, the ribbon material may have a thickness, width and material properties that are sufficiently thin to undergo expansion when the balloon is inflated and undergo deflation when the balloon is deflated. The result is an angioplasty balloon 110 that may be fitted within the armored coil 120. In this example, the armored coil 120 is shown as a ribbon coil that may be pre-wound to the size and shape of the balloon 110 in its expanded state. The ribbon coil may be pre-wound onto a specifically shaped mandrel. Preferably, the armored coil 120 is in the shape of a ribbon coil as shown in FIGS. 1 and 2. Such a geometry provides a balloon catheter 100 assembly that may be flexible as the catheter 100 is maneuvered through the vasculature. Although not shown, the armored coil 120 may be formed from a thin and continuous tubular metal foil or sleeve. Other shapes of the armored coil 120 are contemplated and may be utilized depending on the specific application the balloon catheter 100 is to be used in.

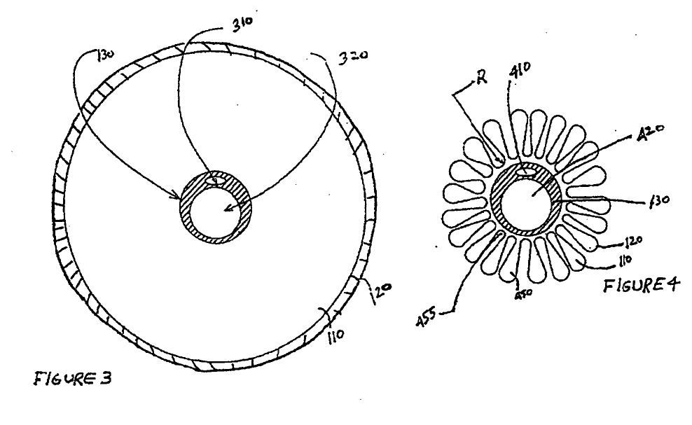

FIG. 3 shows a cross-section of the puncture resistant balloon catheter 100 of FIGS. 1 and 2. The balloon 110 is shown inflated and secured within the armored coil 120. The balloon 110 becomes inflated when inflation fluid is passed through the inflation lumen 310, which extends within the shaft of the catheter 130. As shown, the balloon 10 is fitted within the armored coil 120 such that virtually no gap may be present. Such a fitting may help to reinforce the balloon 110.

FIG. 4 depicts a cross-sectional view of the balloon 110 and armored coil 120 in a collapsed, deflated configuration. As shown in FIG. 4, the coil 120 in the deflated configuration may be bent and folded. The balloon 110 and armored coil 120 are shown as one thickness in order to emphasize the tight fit between them. In this example, the deflated configuration has a series of folding blades 450 circumferentially oriented about the shaft of the catheter 130. The folding blades 450 of the armored coil 120 may be folded by a process similar to the folding process utilized for angioplasty balloons, which is known to one of ordinary skill in the art. Although not shown in FIG. 4, the folding blades 450 may also be wrapped around the catheter as in a conventional balloon catheter.

The folding arrangement enables the puncture resistant balloon catheter 100 to retain a small profile during delivery to the target site. The folding arrangement shown in FIG. 4 may be characterized by a fold radius, R. Suitable values of the fold radius, R, may be dependent upon many factors, including the thickness of the armored coil 120 and the diameter of the catheter 130. Additionally, the fold radius, R, may be selected such that the formed creases 455 are large enough for the balloon 110 to properly expand upon inflation fluid passing into the inflation lumen 410. Nonetheless, because the armored coil 120 is thin with respect to the balloon 110, and the balloon 110 is robust, some plastic deformation may be tolerated at the creases 455. In this example, the fold radius R preferably ranges from about 0.002 inches to about 0.010 inches.

Still referring to FIG. 4, when fluid is passed into the inflation lumen 410, the balloon 110 and armored coil 120 may inflate together to produce the configuration shown in FIG. 3. FIG. 3 indicates that the folding blades 450 are unfolded upon inflation. There is virtually no gap between the inner surface of the armored coil 120 and the outer surface of the balloon 110. Both surfaces may be in contact to produce a configuration in which the balloon 110 is firmly secured within the armored coil 120.

A method of fabrication for the balloon catheter 100 will now be discussed. As mentioned and shown in FIGS. 1 and 2, a preferred embodiment uses a ribbon coil as the armored coil 120, in which the balloon 110 is secured inside the ribbon coil. The thin ribbon coil may be pre-wound to the size and shape of the balloon 110. The armored coil 120 is then placed inside a blow forming mold. The coils of the armored coil 120 may touch the walls of the mold. At this point, a parison of the balloon 110 is placed within the armored coil 120. The parison of the balloon 110 is stretch blow molded inside the armored coil 120 in the conventional manner known to one of ordinary skill in the art. The stretch blow molding blows the balloon 110 out to the interior diameter of the armored coil 120. An adhesive could be applied to the interior surface of the coil so that the coil 120 and balloon 110 adhere together. This adhesive could be a heat activated glue such as a hot melt glue, cyanoacrylate or any other suitable adhesive known to one of ordinary skill in the art. The result is a balloon catheter 100 in which the armored coil 120 encompasses the entire balloon 110. In this embodiment, the natural resting size of the armored coil 120 is the expanded state. The balloon will expand to the natural resting size of the armored coil 120. Upon deflation, the balloon transforms into the pleated folding arrangement, shown in FIG. 4. Because of the relatively thin metal of the ribbon coil 120 as compared to the balloon 110, the armored coil 120 correspondingly collapses into the pleated folding arrangement, shown in FIG. 4.

The armored coil 120 disposed over the balloon catheter 100 may enable high pressure dilating forces. Typical dilating pressures of non-reinforced angioplasty balloons may range from about 15 atmospheres to about 20 atmospheres. Conventional reinforced balloons with fiber or woven Dacron embedded in the balloon material may have dilating pressures of about 50 atmospheres. The addition of a high tensile strength armor such as armored coil 120 disposed over a polymeric balloon such as balloon 110 has the ability to allow dilation pressures as high as about 100 atmospheres.

The ability of the armored coil 120 to reinforce the balloon 110 and allow such high dilating pressures renders the balloon catheter 100 conducive in lumens with highly calcified lesions. Typically, calcifications have the potential for damaging the balloon material of conventional angioplasty balloon catheters. As a result, the balloon inflation procedure may have to be repeated several times before the calcified lesion or blockage will yield. The calcified lesions that need to be expanded in the lumens are generally hard. When a lumen is expanded, the calcified lesions may crack, forming a calcification with sharp edges. The armored coil 120 protects the balloon 110 during expansion of lumens with calcified lesions. This enables balloon expansion of calcified lumens to occur relatively quickly and effectively, without the risk of having to repeat the procedure multiple times because of a balloon puncture.

The armored coil 120 may also protect the balloon 110 from puncture during the implantation of a balloon expandable stent through an opening of a fenestrated graft and into a side branch artery or vessel. A typical implantation procedure may now be described.

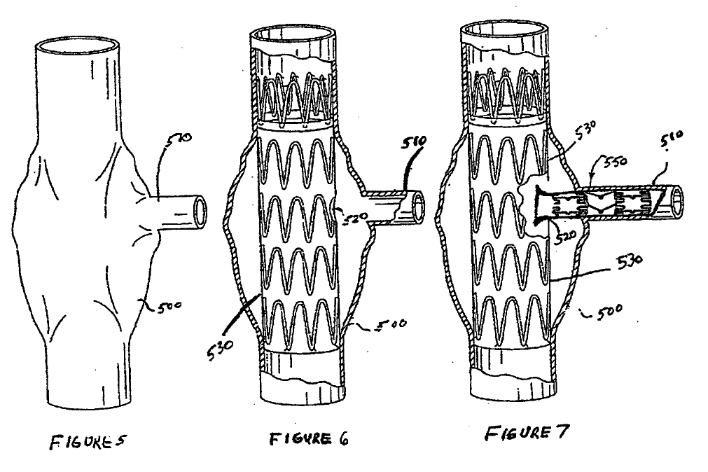

FIG. 5 shows a main lumen 500 and a branch lumen 510. The main lumen 500 has an aneurism, or weakness, which exists where the branch lumen 510 joins the main lumen 500. A stent graft 530, as shown in FIG. 6, maybe implanted within the main lumen 500. Thus, blood flows through the stent graft 530 to alleviate pressure and potential rupture of the weakened wall of the main lumen 500. The stent graft 530 includes a hole or orifice (i.e., fenestration 520) which can be aligned with the branch lumen 510 to allow blood flow to continue through the branch lumen 510 and into the healthy side branch vessels that supply blood to the visceral organs. A blown-up view of the fenestration 520 of the stent graft 530 is shown in FIGS. 8 and 9.

Preferably, there is a tight seal around the fenestration 520 to ensure that blood does not leak out of the space between the stent graft 530 and the wall of the main lumen 500. If blood is allowed to leak into the aneurysm around the area of the fenestration 520, then the aneurysm may continue to be pressurized and a continued risk of rupture may exist. Forming such a seal requires positioning a balloon expandable stent 550 in the branch lumen 510 so that the stent 550 connects the branch lumen 510 to the stent graft 530.

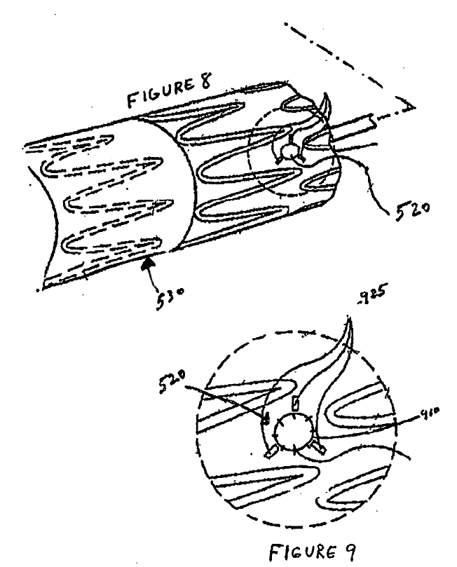

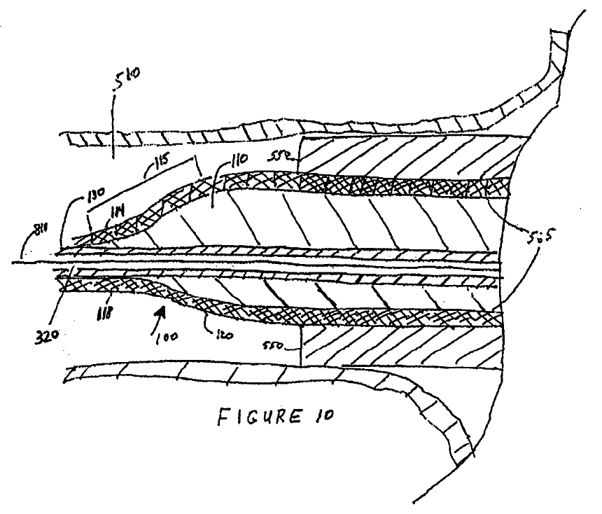

Accordingly, after the stent graft 530 is placed within the main lumen 500 and the fenestration 520 is aligned with the branch lumen 510, the balloon expandable stent 550 may be delivered and deployed. As a result of expansion of the stent 550, it becomes attached to the stent graft 530 through the fenestration 520. Radiopaque markers 925 (FIG. 9) assist with the alignment of the stent 550 into the fenestration 520. The puncture resistant balloon catheter 100 is used to deliver and deploy the balloon expandable stent 550, which is disposed over the armored coil 120. With the stent 550 loaded over the armored coil 120, the puncture resistant balloon catheter 100 may be advanced over a wire guide 810 (FIG. 10) and into the stent graft 530 (FIG. 6). The balloon catheter 100 is maneuvered into the stent graft 530 and then partially through the fenestration 520. Passing inflation fluid through the inflation lumen 310 (FIG. 3) causes the balloon 110 and armored coil 120 to expand from the deflated state to the inflated state. The inflation of the balloon 110 enables the armored coil 120 to expand, which in turn allows the stent 550 to expand within the branch lumen 510, as shown in FIG. 7. The distal end of the stent 550 is disposed within the branch lumen 510. The proximal end of the stent 550 may be flared. The flare acts to anchor the stent 550 against the fenestration 520. At this stage, the stent 550 may be sealed against the fenestration 520 of the stent graft 530 so that blood may flow into the branch lumen 510 without leaking into the aneurysm region.

During implantation of the balloon expandable stent 550 using the balloon catheter 100, there are several instances in the implantation procedure where the balloon 110 may be protected from puncture by the armored coil 120. For example, as the balloon catheter 100 is maneuvered through the fenestration 520 to implant the stent 550, the fenestration 520 may puncture the balloon 110. FIGS. 8 and 9 show the fenestration 520 in greater detail. FIG. 9 shows a nitinol circumferential ring of wire 910 that is sutured to the graft material around the fenestration 520. The nitinol circumferential ring of wire 910 strengthens the fenestration 520, allowing for a more stable fixation when the balloon expandable stent 550 is connected. A lack of wire 910 may cause the positions of the fenestration 520 to be less reliable and may make it more difficult to seal the stent 550. As the balloon catheter 100 is maneuvered through the fenestration 520 to deploy the stent 550, preferably with the assistance of radiopaque markers 925 (FIG. 9), the nitinol circumferential ring of wire 910 may contact the surface of the balloon 110, thereby potentially rupturing a conventional balloon. The armored coil 120 may prevent the wire 910 from damaging and potentially rupturing the balloon 110.

Additionally, balloon puncture may occur as the balloon expandable stent 550 is being inflated within the branch lumen 510. More specifically, the proximal end of the balloon 110 is preferably flared in order to ensure a tight seal between the stent graft 530 and the branch lumen 510. This flaring process may turn some of the ends of the struts of the stent 550 inward. Such a configuration may penetrate and rupture the balloon 110. Accordingly, the armored coil 120 may prevent the flared struts of the expanded stent 550 from rupturing the balloon 110.

FIG. 10 shows the puncture resistant balloon catheter 100 in an expanded state. FIG. 10 is a cross-sectional view of the balloon catheter 100 along its longitudinal axis. With the aid of a wire guide 810 through a wire guide lumen 320, a portion of the balloon catheter 100 may be maneuvered through the fenestration 520 and thereafter be expanded to deploy a distal portion of the stent 550 within the branched lumen 510. The stent 550 may be delivered using a delivery sheath to keep it from being expanded. The delivery sheath can be withdrawn before expanding the stent 550. With the delivery sheath withdrawn, the stent 550 is shown expanded and disposed over the armored coil 120. The stent 550 is expanded by inflating balloon 110. The balloon 110 becomes inflated when inflation fluid is passed through the inflation lumen 310. In this example, the armored coil 120 also covers the tapered end 115 of the balloon 110. The armored coil 120 is formed with tapered ends 114, 119 that may conform with the tapered ends 115 of the balloon 110. Additionally, a portion of the interior of the armor coil 120 may be coated with an adhesive 565 to further secure the armored coil 120 to the surface of the balloon 110. The inflation of the balloon 110 may enable the armored coil 120 to expand, which in turn may allow the stent 550 to expand within branched lumen 510. The armored coil 120 may protect the balloon 110 from puncturing during the implantation of the stent 550.

The above figures and disclosure are intended to be illustrative and not exhaustive. This description will suggest many variations and alternatives to one of ordinary skill in the art. All such variations and alternatives are intended to be encompassed within the scope of the attached claims. Moreover, the advantages described herein are not necessarily the only advantages of the invention, and not all of the described advantages will be necessarily achieved with every embodiment of the invention. Those familiar with the art may recognize other equivalents to the specific embodiments described herein which equivalents are also intended to be encompassed by the attached claims.

1. A puncture resistant balloon catheter comprising:
a catheter comprising a shaft extending along a longitudinal axis of the catheter and an inflation lumen extending therethrough;
a balloon overlying the shaft of the catheter, the inflation lumen being in communication with the balloon to inflate the balloon; and
a puncture resistant cover disposed over the balloon, the cover extending circumferentially around the balloon along a length thereof, the cover adapted to be movable between a deflated state and an inflated state in response to inflation of the balloon, wherein the cover inhibits piercing of the balloon.

2. The puncture resistant balloon catheter of claim 1, wherein the puncture resistant cover reinforces the inflatable balloon, further wherein the puncture resistant cover is adapted to provide dilating pressures up to about 100 atmospheres.

3. The puncture resistant balloon catheter of claim 1, wherein the puncture resistant cover is a ribbon coil.

4. The puncture resistant balloon catheter of claim 1, wherein the puncture resistant cover is a sleeve.

5. The puncture resistant balloon catheter of claim 1, wherein the cover comprises a metallic alloy.

6. The puncture resistant balloon catheter of claim 1, wherein at least a portion of an inner surface of the puncture resistant cover is coated with an adhesive to secure the inner surface of the cover to the balloon.

7. The puncture resistant balloon catheter of claim 1, in combination with a balloon expandable stent in a generally compressed configuration overlying a portion of the puncture resistant cover.

8. The puncture resistant balloon catheter of claim 1, wherein the balloon and the cover in a deflated state are configured in a folding arrangement, the folding arrangement comprising a predetermined number of pleats positioned circumferentially around the shaft of the catheter.

9. The puncture resistant balloon catheter of claim 8, wherein the pleats are characterized by a folding radius, the folding radius ranging from about 0.002 inches to about 0.010 inches.

10. The puncture resistant balloon catheter of claim 1, wherein the inflated state is characterized by an absence of pleats, and an outer surface of the balloon is secured to an inner surface of the cover.

11. A delivery system for deploying a prosthesis, comprising:
a catheter comprising a shaft extending along a longitudinal axis of the catheter and an inflation lumen extending therethrough;
a balloon overlying the shaft of the catheter, the inflation lumen being in communication with the balloon to inflate the balloon;
a puncture resistant coil disposed over the balloon, the coil extending circumferentially around the balloon along a length thereof, the coil adapted to be movable between a deflated state and an inflated state in response to inflation of the balloon, wherein the coil inhibits piercing of the balloon; and
a balloon expandable stent overlying a portion of the coil.

12. The delivery system of claim 11, wherein the coil is in a generally deflated configuration with the balloon.

13. The delivery system of claim 11, wherein the coil has a thickness ranging from about 0.0001 inches to about 0.002 inches and a width ranging from about 0.010 inches to about 0.040 inches.

14. The delivery system of claim 11, wherein the coil is wound to the size and shape of the balloon.

15. The delivery system of claim 11, wherein the coil comprises one or more tapered ends that conform to one or more tapered portions of the balloon.

16. A method of deploying a balloon expandable stent within a branched body lumen through a fenestration of a graft comprising the steps of:
(a) providing a puncture resistant balloon catheter comprising:
(i) a catheter comprising a wire guide lumen and inflation lumen extending therethrough, an inflatable balloon overlying the catheter, and a puncture resistant cover disposed over the balloon, the cover extending circumferentially around the longitudinal axis of the catheter;
(ii) providing a wire guide, the wire guide extending through the wire guide lumen of the catheter;
(iii) providing a balloon expandable stent, the stent being disposed over the puncture resistant cover in a generally compressed configuration;
(b) advancing the puncture resistant balloon catheter over the wire guide, the balloon catheter being in the deflated state;
(c) advancing the puncture resistant balloon catheter into the graft, the fenestration of the graft being aligned with the branched body lumen;
(d) maneuvering the puncture resistant balloon catheter through the fenestration of the graft and into the branched body lumen; and
(e) passing fluid through the inflation lumen to inflate the balloon, wherein the inflation of the balloon causes the cover to transform from a deflated state to an expanded state, the cover exerting an outward force to expand the stent against one or more walls of the branched body lumen, a distal end of the stent extending within the branched body lumen and a proximal end of the stent extending through the fenestration of the graft.

17. The method of claim 16, wherein the cover inhibits puncture of the balloon as struts of the stent extend into a flared configuration and contact a surface of the puncture resistant cover.

18. The method of claim 16, wherein the puncture resistant cover inhibits puncture of the balloon as the balloon catheter passes through the fenestration, the fenestration comprising a rim of wire.

19. The method of claim 16, further comprising the step of navigating the puncture resistant balloon catheter into an abdominal aorta.

20. The method of claim 16, further comprising the step of navigating the puncture resistant balloon catheter into a thoracic aorta.
